(19) United States
(12) Patent Application Publication
(10) Pub. No.: US 2004/0091551 A1
(43) Pub. Date: May 13, 2004

(54) Topical composition and application system

(76) Inventor: Al-Karim Damji, Thornhill (CA)

Correspondence Address: Michael S. Neustel, Suite No. 4, 2534 South University Drive, Fargo, ND, 58103, US

(21) Appl. No.: 10/294,500

(22) Filed: Nov. 13, 2002

(51) Int. Cl. A61K 33/32

(52) U.S. Cl. 424/642

(57) ABSTRACT

A topical composition and application system for effectively and efficiently treating and/or preventing diaper dermatitis. The composition includes an amount of active medicinal ingredient, one or more non-medicinal ingredients used for enhancing the utility of the topical composition, and a carrier for application through either a spray-type applicator or impregnated upon a pre-moistened disposable towelette. The active ingredient is preferably zinc oxide ranging from 5 to 50% by weight, although any other active ingredient used for the prevention and/or treatment of diaper dermatitis may be used. The non-medicinal agent used may include any of the following: dimethicone, lanolin, mineral oil, petrolatum, antimicrobial agents, urea, calcium undecylenate, talc, starch, calcium carbonate, cod liver oil, Aloe, vitamin A, vitamin D, vitamin E, benzalkonium chloride, stearic acid, cetyl alcohol, skin soothing agents, emollients, moisteners, humectants, powders, pH stabilizers, chelating agents and emulsifiers.

### CROSS REFERENCE TO RELATED APPLICATIONS

Not applicable to this application.

### STATEMENT REGARDING FEDERALLY SPONSORED RESEARCH OR DEVELOPMENT

Not applicable to this application.

### BACKGROUND OF THE INVENTION

1. Field of the Invention

The present invention relates generally to topical compositions and more specifically it relates to a topical composition and application system for effectively and efficiently treating and/or preventing diaper dermatitis.

Diaper dermatitis, commonly referred to diaper rash, is a contact dermatitis and can occur to any individual who uses a diaper. It is the most common skin disorder for young children, but may affect incontinent and paralyzed individuals who wear diapers. Diaper dermatitis is an extremely common condition of the perineal area, including the perineum, buttocks, perinal, coccyx and upper and inner thighs. This condition can present with any or all of the following redness, maceration, swelling, vesiculation, crusting, scaling, pigmentary changes, excoriation and thickening.

Diaper dermatitis develops from a combination of moisture, heat, friction and caustic build up and prolonged contact of human waste products to the skin. Since diaper dermatitis causes a breakdown of the protective skin barrier, it is predisposed to being superinfected by the normal skin commensals like bacteria and organisms normally found in fecal excrements like candida. If left untreated, diaper dermatitis can cause further damage to the skin with increased inflammation and infection. Though rare, there is the possibility of systemic infection, which can be life threatening. Due to the multifactorial nature of diaper dermatitis, the most effective way of treating and preventing this condition has been the application of a topical skin barrier to the affected area. This skin barrier prevents the body waste from prolonged contact with the sensitive skin.

### DESCRIPTION OF THE RELATED ART

Topical barrier compositions have been commercially available for years for treating diaper dermatitis. Typically, these barrier compositions come in the form of creams and ointments and they contain a variety of ingredients. These ingredients include starch, petrolatum, lanolin, silicone, zinc oxide and the like. These ingredients are normally applied to the affected area by sprinkling the ingredients onto the affected area or by rubbing the material onto the skin. Often these topical barriers are stored within multi-dosage containers such as flexible tubular containers, cans, tubs and the like which predispose the contents to inadvertent contamination through repeated uses.

Wiping materials are commonly used for cleansing the affected area before application of the topical barrier. These wiping materials are usually pre-moistened disposable towelettes made from nonwoven fibers. Therefore, the common procedure in changing a diaper is to remove the diaper, use a cleaning method to clean the affected area, and then treat the area with another method for applying the topical barrier.

Examples of patented devices which may be related to the present invention include U.S. Pat. No. 6,352,700 to Luu et al.; U.S. Pat. No. 5,869,071 to Wieselman et al.; U.S. Pat. No. 5,648,083 to Blieszner et al.; U.S. Pat. No. 5,043,155 to Puchalski et al.; U.S. Pat. No. 5,256,417 to Koltisko; U.S. Pat. No. 6,331,295 to Schulz; U.S. Pat. No. 6,361,784 to Brennan et al.; U.S. Pat. No. 3,029,187 to Steinhardt; U.S. Pat. No. 4,867,970 to Newsham et al.; U.S. Pat. No. 4,948,580 to Browning; U.S. Pat. No. 5,002,938 to Wang et al.; U.S. Pat. No. 5,110,809 to Wang et al.; U.S. Pat. No. 5,174,475 to Day et al.; U.S. Pat. No. 5,219,877 to Shah et al.; U.S. Pat. No. 5,330,452 to Zook; U.S. Pat. No. 5,415,866 to Zook; U.S. Pat. No. 5,446,070 to Mantelle; U.S. Pat. No. 5,622,993 McGinity et al.; U.S. Pat. No. 5,686,088 to Mitra et al.; U.S. Pat. No. 5,686,089 to Mitra et al.; U.S. Pat. No. 5,714,165 to Repka et al.; U.S. Pat. No. 5,719,197 to Kanios et al.; U.S. Pat. No. 5,853,732 to Munden; and U.S. Pat. No. 6,056,954 to Fischetti et al.

While this prior art may be suitable for the particular purpose to which they address, they are not as suitable for effectively and efficiently treating and/or preventing diaper dermatitis by combining the multiple steps involved in the care and treatment of diaper dermatitis into one step using an active medicinal agent. While the prior art has provided compositions having various levels of utility in personal cleansing or in the treatment for the prevention of diaper dermatitis, the prior art has not provided the compositions in the manner or the extent proposed in present invention. In addition, current methods for the application of the topical barrier normally include the requirement to come into physical contact with the affected area. A method to apply a topical barrier without physical contact is not only more hygienic for the caregiver and the affected individual, but also it prevents the inadvertent contamination of the topical composition.

In these respects, the topical composition and application system according to the present invention substantially departs from the conventional methods of use and compositions of the prior art, and in so doing provides a composition and methods of using the composition primarily developed for the purpose of effectively and efficiently treating and/or preventing diaper dermatitis.

### BRIEF SUMMARY OF THE INVENTION

In view of the foregoing disadvantages inherent in the known types of topical compositions now present in the prior art, the present invention provides a new topical composition and application system wherein the same can be utilized for effectively and efficiently treating and/or preventing diaper dermatitis.

The general purpose of the present invention, which will be described subsequently in greater detail, is to provide a new topical composition and application system that has many of the advantages of the topical compositions mentioned heretofore and many novel features and functions that result in a new topical composition and application system which is not anticipated, rendered obvious, suggested, or even implied by any of the prior art topical compositions, either alone or in any combination thereof.

To attain this, the present invention generally comprises combining a medicinal ingredient, such as zinc oxide, with non-medicinal ingredients to enhance the use of the topical composition with a carrier medium for application with a sprayer, or impregnated into a wiping material.

There has thus been outlined, rather broadly, the more important features of the invention in order that the detailed description thereof may be better understood, and in order that the present contribution to the art may be better appreciated. There are additional features of the invention that will be described hereinafter and that will form the subject matter of the claims appended hereto.

In this respect, before explaining at least one embodiment of the invention in detail, it is to be understood that the invention is not limited in its application to the details of construction and to the arrangements of the components set forth in the following description or illustrated in drawings. The invention is capable of other embodiments and of being practiced and carried out in various ways. Also, it is to be understood that the phraseology and terminology employed herein are for the purpose of the description and should not be regarded as limiting.

A primary object of the present invention is to provide a topical composition and application system that will overcome the shortcomings of the prior art.

Another object is to provide a topical composition and application system that effectively treats and/or prevents diaper dermatitis.

A further object is to provide a topical composition and application system that is easy to apply to an affected area upon the skin via direct application or with a spray container.

Yet another object is to provide a topical composition and application system that provides a unique method for expediting the diaper changing process by providing the effective composition impregnated into a towelette such that the process of pre-wiping the affected soiled area and application of the composition is done in a single step without compromising the utility of either.

Another object is to provide a topical composition and application system that is convenient, clean, hygienic, and portable to utilize.

An additional object is to provide a topical composition and application system that reduces the steps necessary to treat or prevent diaper dermatitis.

Other objects and advantages of the present invention will become obvious to the reader and it is intended that these objects and advantages are within the scope of the present invention.

To the accomplishment of the above and related objects, this invention may be embodied in the form illustrated in the accompanying drawings, attention being called to the fact, however, that the drawings are illustrative only, and that changes may be made in the specific use illustrated and described within the scope of the appended claims.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 is an upper perspective view of an exemplary spray applicator.

FIG. 2 is a side view of the exemplary spray applicator.

FIG. 3 is an upper perspective view of an exemplary dispenser.

FIG. 4 is an exploded upper perspective view of an exemplary dispenser.

FIG. 5 is an upper perspective view of an exemplary pouch.

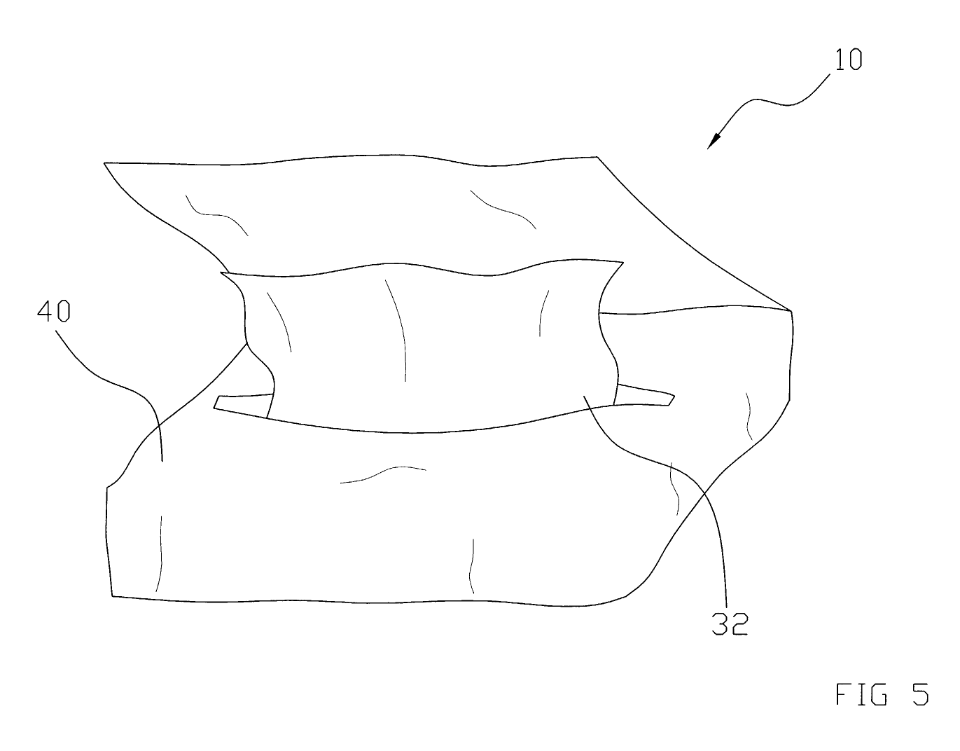

### DETAILED DESCRIPTION OF THE INVENTION

A. Topical Composition

The topical composition is preferably comprised of an amount of active medicinal ingredient ranging from between 1 and 50%, a protective barrier and an amount of carrier making up the remaining volume of the topical composition. All percentages may be based upon either weight or volume. Additional non-medicinal ingredients may be added to either enhance the utility of the topical composition or to prolong its shelf life. However, these non-medicinal ingredients are not required. Examples of these types of non-medicinal ingredients could include fragrance, dimethicone, lanolin, mineral oil, petrolatum, antimicrobial agents, urea, calcium undecylenate, talc, starch, calcium carbonate, cod liver oil, Aloe, vitamin A, vitamin D, vitamin E, benzalkonium chloride, stearic acid, cetyl alcohol, skin soothing agents, emollients, moisteners, humectants, powders, pH stabilizers, chelating agents and emulsifiers. However it can be appreciated by one skilled in the art that other currently available non-medicinal ingredients could be included to enhance the utility or prolong the shelf life of the topical composition.

1. Carrier

The carrier may be comprised of any substance that does not negatively affect the efficacy of the active medicinal ingredient. For example the carrier may be comprised of water, lotion, cream, ointment or powder capable of being transmitted effectively to the affected area. The carrier is preferably a fluid, powder or cream that is capable of being sprayed or applied upon an affected area from a conventional applicator system.

2. Active Medicinal Ingredient

The preferred formulation of the topical treatment composition is comprised of at least 5% and up to approximately 50% zinc oxide as the active medicinal ingredient. Particularly preferred compositions contain from at least 5% to less than 40%, more preferably at least 5% to 35%, and most preferably from about 15% to 30% zinc oxide.

B. Applicators

1. Disposable Towelettes

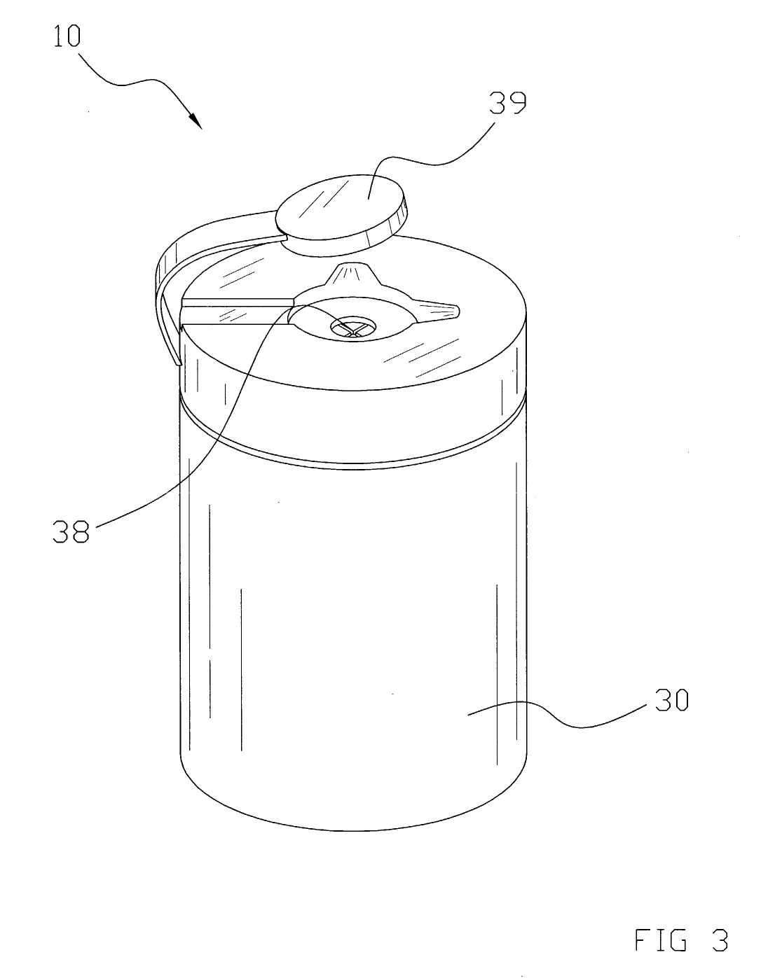

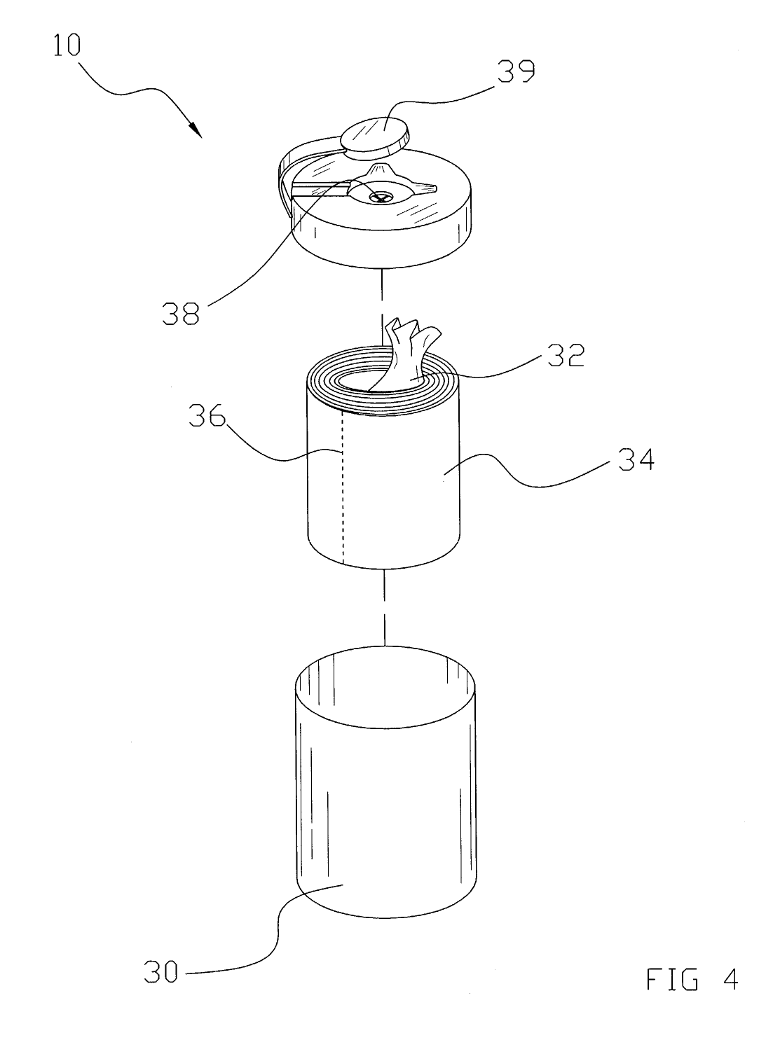

An embodiment of the present invention could include the topical composition being impregnated into a personal hygiene disposable towelette. Current commercially available pre-moistened infant wipes could be one available medium. As shown in FIG. 3 and 4, impregnated pre-moistened medicated disposable towelettes 32 would be preferably stored in a roll 34 and placed in a dispenser 30. This roll 34 of impregnated pre-moistened medicated disposable towelettes 32 would preferably have perforations 36 at regular intervals. The roll 34 of impregnated pre-moistened medicated disposable towelettes 32 would be threaded through the central opening 38 of the dispenser 30. The dispenser 30 would include a lid 39 that could be placed over the central opening 38 to prevent soiling and drying of the remaining stored impregnated pre-moistened medicated disposable towelettes 32. Another embodiment of the present invention could include individual impregnated pre-moistened medicated disposable towelettes 32 stored in pouch 40 cable of being repeatedly opened and repeatedly sealed shut. However, it can be appreciated by one skilled in the art that other methods of storing and dispensing impregnated pre-moistened medicated disposable towelettes 32 could be utilized.

2. Spray

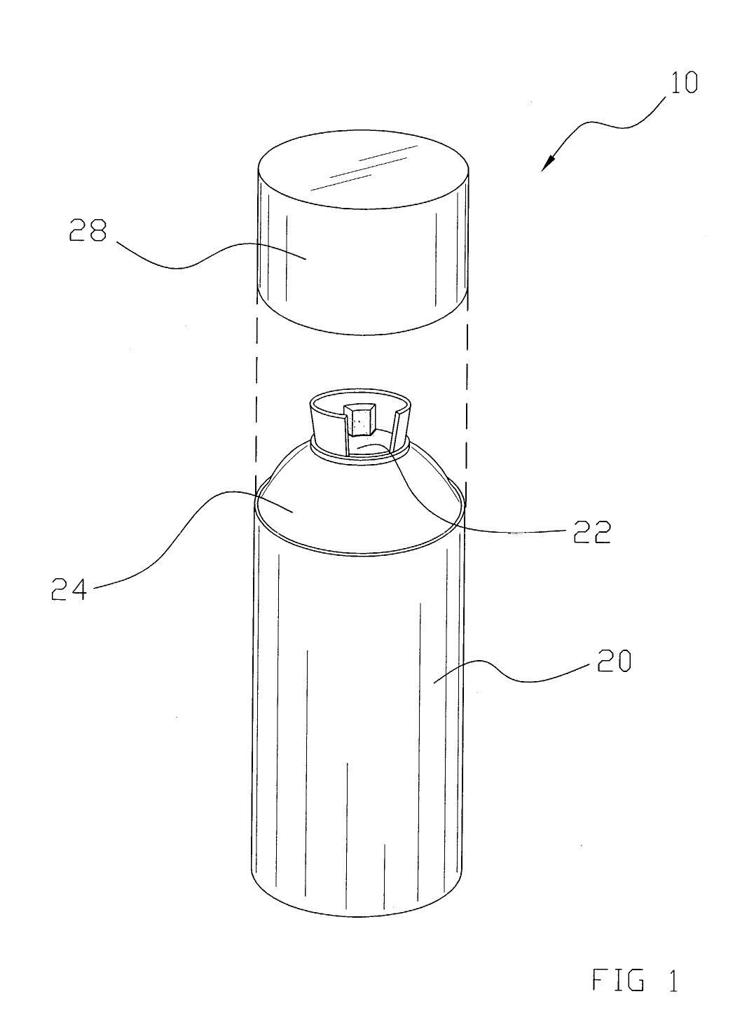

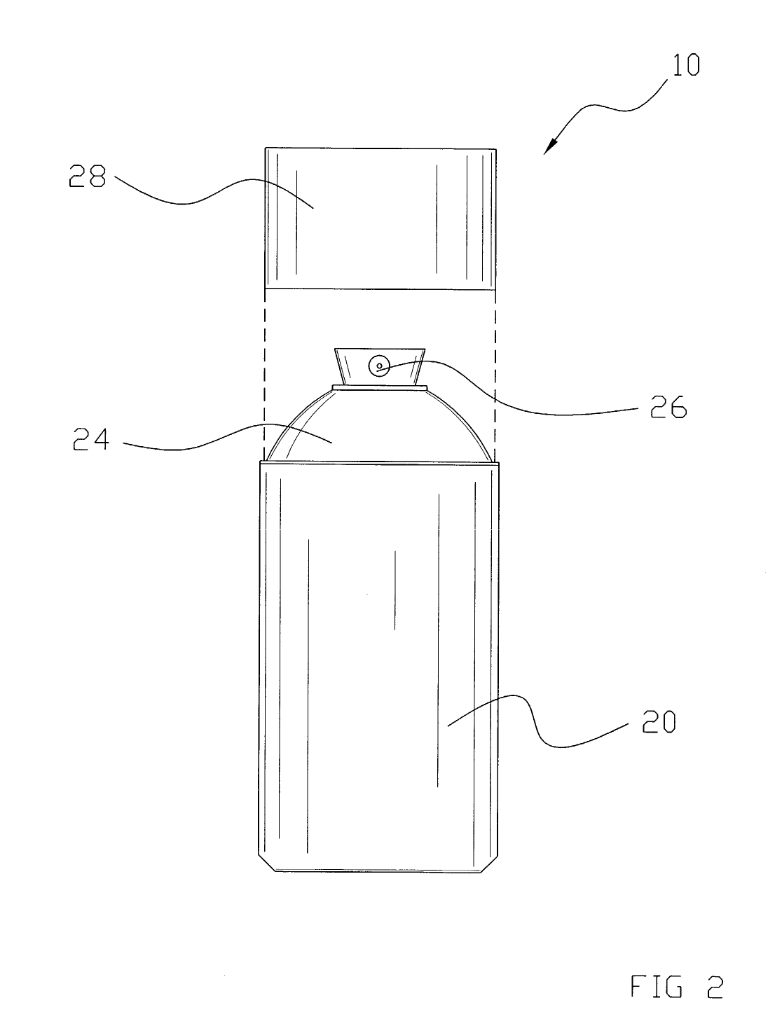

In another embodiment the topical composition may be stored within and dispensed from a conventional spray container or similar dispensing device (such as a “pump bottle” or the like) that does not require the individual to touch the topical composition within the container 20 directly with their hands. An exemplary applicator is illustrated within FIGS. 1 and 2 of the drawings, however it can be appreciated that various other types of applicator devices that are currently employed to dispense fluid, powders and lotions may be utilized.

As shown in FIGS. 1 and 2 of the drawings, the exemplary spray container preferably is comprised of a container 20 for storing a volume of topical composition, an upper portion 50 having an upper end 40 within said container 20, and a nozzle 60 fluidly connected within the container 20 for dispensing the topical composition from the container 20. A cap 28 may be included to protect the nozzle 60. The container 20 is preferably pressurized to allow for easy dispensing of the topical composition from the container 20. The embodiment of the present invention with the contents contained in a “pump bottle” would preferably not be under pressure.

C. Methods of Application

1. Disposable Towelettes

In use the impregnated pre-moistened medicated disposable towelettes 32 would be used once the diaper is removed. In one embodiment a impregnated pre-moistened medicated disposable towelette 32 would be drawn from the dispenser 30 and detached at the next perforation 36 on the roll 34 from the remainder of the roll 34 of disposable towelettes 32. The central opening 38 of the dispenser 30 is preferably designed to facilitate detaching one impregnated pre-moistened medicated disposable towelette 32 from the roll 34 at a time. The impregnated pre-moistened medicated disposable towelette 32 would be simultaneously used to clean the affected area while applying the topical composition for the treatment and/or prevention of diaper dermatitis.

2. Spray

In use the topical composition could be sprayed on the affected area after the affected area has been cleaned. Once the topical composition is applied to the affected area, the user may further rub the applied topical composition upon the affected skin area or simply allow the topical composition to remain upon the affected skin area.

As to a further discussion of the manner of usage of the present invention, the same should be apparent from the above description. Accordingly, no further discussion relating to the manner of usage will be provided.

With respect to the above description then, it is to be realized that the optimum relationships for the components of the invention, to include variations in proportions and manner of use are deemed readily apparent and obvious to one skilled in the art.

Therefore, the foregoing is considered as illustrative only of the principles of the invention. Further, since numerous modifications and changes will readily occur to those skilled in the art, it is not desired to limit the invention to the exact composition and use shown and described, and accordingly, all suitable modifications and equivalents may be resorted to, falling within the scope of the invention.

I claim:

1. A topical composition and application system for treating and/or preventing diaper dermatitis, comprising:
an active medicinal agent;
a non-medicinal agent; and
a carrier for receiving said active medicinal agent and said non-medicinal agent.

2. The topical composition and application system for treating and/or preventing diaper dermatitis of claim 1, wherein said active medicinal agent is zinc oxide.

3. The topical composition and application system for treating and/or preventing diaper dermatitis of claim 2, wherein said zinc oxide is approximately 5 to 50% by weight.

4. The topical composition and application system for treating and/or preventing diaper dermatitis of claim 1, wherein said non-medicinal agent is selected from the group consisting of silicone, dimethicone, lanolin, mineral oil, petrolatum, antimicrobial agents, urea, calcium undecylenate, talc, starch, calcium carbonate, cod liver oil, Aloe, vitamin A, vitamin D, vitamin E, benzalkonium chloride, stearic acid, cetyl alcohol, skin soothing agents, emollients, moisteners, humectants, powders, pH stabilizers, chelating agents and emulsifiers.

5. A topical composition and application system for treating and/or preventing diaper dermatitis, comprising:
an active medicinal agent;
a plurality of non-medicinal agents; and
a carrier for receiving said active medicinal agent and said non-medicinal agent.

6. The topical composition and application system for treating and/or preventing diaper dermatitis of claim 5, wherein said active medicinal agent is zinc oxide.

7. The topical composition and application system for treating and/or preventing diaper dermatitis of claim 6, wherein said zinc oxide is approximately 5 to 50% by weight.

8. The topical composition and application system for treating and/or preventing diaper dermatitis of claim 5, wherein said non-medicinal agents are selected from the group consisting of silicone, dimethicone, lanolin, mineral oil, petrolatum, antimicrobial agents, urea, calcium undecylenate, talc, starch, calcium carbonate, cod liver oil, Aloe, vitamin A, vitamin D, vitamin E, benzalkonium chloride, stearic acid, cetyl alcohol, skin soothing agents, emollients, moisteners, humectants, powders, pH stabilizers, chelating agents and emulsifiers.

9. The topical composition and application system for treating and/or preventing diaper dermatitis of claim 5, including an applicator device for applying said topical composition comprising:
a container capable of storing a volume of said topical composition; and
a spray head having a lever and a spray nozzle for dispensing said topical composition upon an affected area.

10. The topical composition and application system for treating and/or preventing diaper dermatitis of claim 9, wherein said container may be pressurized.

11. The topical composition and application system for treating and/or preventing diaper dermatitis of claim 9, wherein said spray head is mechanically actuated.

12. The topical composition and application system for treating and/or preventing diaper dermatitis of claim 5, wherein said topical composition is impregnated into a pre-moistened personal hygiene disposable towelette.

13. A topical composition and application system for treating and/or preventing diaper dermatitis, comprising:
from about 5 to 50% by weight zinc oxide;
a plurality of non-medicinal agents; and
a carrier for receiving said active medicinal agent and said non-medicinal agent.

14. The topical composition and application system for treating and/or preventing diaper dermatitis of claim 13, wherein said non-medicinal agents are selected from the group consisting of silicone, dimethicone, lanolin, mineral oil, petrolatum, antimicrobial agents, urea, calcium undecylenate, talc, starch, calcium carbonate, cod liver oil, Aloe, vitamin A, vitamin D, vitamin E, benzalkonium chloride, stearic acid, cetyl alcohol, skin soothing agents, emollients, moisteners, humectants, powders, pH stabilizers, chelating agents and emulsifiers.

15. The topical composition and application system for treating and/or preventing diaper dermatitis of claim 13, including an applicator device for applying said topical composition comprising:
a container capable of storing a volume of said topical composition; and
a spray head having a lever and a spray nozzle for dispensing said topical composition upon an affected area.

16. The topical composition and application system for treating and/or preventing diaper dermatitis of claim 15, wherein said container may be pressurized.

17. The topical composition and application system for treating and/or preventing diaper dermatitis of claim 15, wherein said spray head is mechanically actuated.

18. The topical composition and application system for treating and/or preventing diaper dermatitis of claim 13, wherein said topical composition is impregnated into a pre-moistened personal hygiene disposable towelette.
